(19) United States
(12) Patent Application Publication
(10) Pub. No.: US 2009/0216337 A1
(43) Pub. Date: Aug. 27, 2009

(54) GASTRIC BYPASS PROSTHESIS FIXATION SYSTEM AND METHOD FOR TREATMENT OF OBESITY

(75) Inventors: Thomas D. Egan, Marblehead, MA (US); George Adaniya, Wellesley, MA (US)

Correspondence Address: FOLEY & LARDNER LLP, 111 HUNTINGTON AVENUE, 26TH FLOOR, BOSTON, MA, 02199-7610, US

(73) Assignee: Gastrix Medical, LLC

(21) Appl. No.: 11/909,024

(22) PCT Filed: Mar. 17, 2006
(86) PCT No.: PCT/US06/10050

§ 371 Date: Jun. 23, 2008

Related U.S. Application Data
(60) Provisional application No. 60/662,988, filed on Mar. 18, 2005.

(51) Int. Cl. A61F 2/04

(52) U.S. Cl. 623/23.64; 604/8

(57) ABSTRACT

An improved gastric bypass prosthetic (GBP) device and an improved GBP fixation device, as well as a GBP kit for the surgical treatment of obesity. In one form, a gastric bypass prosthetic (GBP) device of the invention includes a optional flexible tube extending along a central axis from a proximal end to a distal end, and having a tubular tissue fixation portion at or near said proximal end. The tissue fixation portion extends along the central axis between a proximal end and a distal end of the tissue fixation portion, and has an annular outer surface between the proximal end and the distal end of the tissue fixation portion.

This application claims priority to U.S. Provisional Application No. 60/662,988, filed on Mar. 18, 2005.

### FIELD OF THE INVENTION

The present invention relates to surgical treatments for morbid obesity and safer alternatives to conventional surgical approaches to obesity treatment.

### BACKGROUND OF THE INVENTION

In the past 25 years the obesity rate doubled for U.S. adults and tripled for U.S. adolescents. The health risks of obesity (diabetes, heart disease, etc.) are well documented, as are the impacts on society in terms of health care cost, insurance coverage, worker productivity and more. Several treatment options are available, low calorie diet, increased physical activity, behavior modification, pharmacotherapy and bariatric surgery. Of these, bariatric surgery has proven to be the most effective treatment option available.

The evolution of surgical treatments for obesity has yielded two dominant forms as of this writing; Roux en Y Gastric Bypass (RGB), and Adjustable Gastric Banding (AGB). (RGB is an evolved refinement of various forms of gastric bypass surgery performed since the 1950s. AGB is based on an implanted gastric band invented by Kuzmak, et al, U.S. Pat. No. 4,592,339.) RGB entails radical surgical reconfiguration of the digestive tract. It is the most effective treatment for obesity available, however it is a complicated, time consuming and expensive operation. RGB also suffers from an unacceptably high incidence of post surgical complications (5% to 7% re-hospitalization rate), and mortality (0.5% death rate from surgical complications). AGB has proven to be a less invasive, less costly, and less dangerous alternative to RGB, but is less effective as a weight loss therapy. (Typical excess weight loss [EWL] at 12 months: RGB=64%, AGB=39%, EWL at 24 months: RGB=71%, AGB=46%.) The differences in efficacy are due to the differences in physiological response to the two procedures. The principal responses to RGB are volumetric restriction of caloric intake (due to small 50 ml stapled stomach pouch created by dividing stomach), malabsorption of ingested food (due to bypassing the distal stomach and 100 cm to 200 cm of proximal intestine, and the diversion of digestive fluids including gastric acid, bile and pancreatic fluids to distal introduction into the food stream), and appetite suppression through inhibition of production of the appetite stimulant hormone ghrelin (due to isolation of the ghrelin producing tissues in the bypassed stomach and duodenum from contact with nutrients.) By contrast, AGB works through volumetric restriction of caloric intake alone (Banding produces a reduced volume pouch in the proximal stomach, but does not achieve malabsorption or ghrelin suppression to the same degree as RGB.

To address the need for an obesity treatment that is as effective as RGB, yet as safe, less-invasive and cost effective as AGB, one of the present inventors has previously disclosed the Gastric Bypass Prosthesis (GBP), (Egan, WIPO Pat. Application #WO 03/094785 A1). The above referenced GBP achieves all the physiological responses of RGB (restriction, malabsorption, diversion and suppression) without major surgery or reconfiguration of the GI tract. The GBP, as described, is delivered through the throat and divides the stomach into two chambers through the use of sutures or staples deployed within the lumen of the stomach for fixation of the device to the stomach wall. In practice, secure, leak-tight fixation using sutures or staples in the stomach delivered through the throat is difficult to achieve, and subject to the possibility of stitch-line infection, migration and breakdown where the suture or staple line introduces a pathway for bacteria from the septic inner lumen of the stomach, through the mucosa, and into the vascular regions of the submucosal and muscularis layers. We will therefore disclose an improved technique and apparatus for GBP fixation, as well as a new and improved therapeutic approach for the treatment of morbid obesity, which will become apparent to those skilled in the art through the following objects and specifications of the present invention.

It is an object of the present invention to provide a method and apparatus for fixating a GBP in the stomach of a patient that eliminates the need for intralumenal suturing or stapling.

It is another object of the present invention to provide a method and apparatus for fixating a GBP in the stomach of a patient that is secure and leak-tight.

It is another object of the present invention to provide a GBP that can be introduced through the esophagus of a patient without injury.

It is yet a further object of the present invention to provide a GBP that can be removed through the esophagus of a patient without injury.

It is a further object of the present invention to provide a method and apparatus for fixating a GBP in the stomach of a patient that resists stomach wall injury from infection due to mucosal penetration, ulceration and contact pressure necrosis.

It is another object of the present invention to provide a method and apparatus for fixating a GBP in the stomach of a patient using commercially available AGBs.

It is another object of the present application to provide a treatment for obesity that is restrictive only and does not require post-implantation adjustment of the stoma.

It is yet a further object of the present invention to provide a treatment regimen for an obese patient that is adaptable to the changing needs of the patient without invasive surgery.

It is a further object of the present invention to provide a treatment regimen for an obese patient that can be used in combination or conjunction with established treatment methodology for maximum benefit for the patient.

### SUMMARY OF THE INVENTION

The forgoing objects are met in new and improved devices and methods for the treatment of obesity. The above referenced GBP is known to the art. Similarly, AGBs and prior art simple non-adjustable gastric bands are known to the art. These bands, when properly implanted, usually through laparoscopic surgery, are known to maintain the formation of restriction within the stomach. Bands are typically held in place through folding and stitching the serosa of the fundus around the band to prevent slippage. Band shape and material selection are designed to be atraumatic to prevent serosal irritation that may result in inflammation, scarring, and eventual encapsulation and migration of the band through the stomach wall. A key feature of AGBs and simple bands is their ability to precisely regulate the size of the stoma (anatomical exit) of the stomach pouch in order restrict caloric intake of the patient. Too large a stoma would cause food to move through the pouch and into the distal stomach too rapidly, allowing the patient to eat unrestricted meal sizes and the absence of a feeling of satiety or fullness at the completion of a small meal. Too small a stoma results in the inability of the pouch to empty, denying the patient nutrition and resulting in vomiting.

One component of the present invention is a GBP fixation device that shares some aspects with prior art gastric bands, but differs in several important aspects. Like conventional AGBs and simple bands, the GBP fixation device is designed to be delivered through open or (preferably) laparoscopic surgery, encircle the stomach between the esophagus and the pylorus, be atraumatic, and be held in place using over-stitching techniques. However, unlike conventional gastric bands which are designed to control the size and shape of an unsupported stoma, the GBP fixation device is designed to suspend the GBP within the lumen of the stomach, apply sufficient pressure to hold the stomach wall against the GBP to maintain a leak-tight seal, and conform to the shape of the GBP to distribute contact pressure over a large enough area to prevent contact pressure necrosis and ulceration.

In one embodiment of the GBP fixation device, the sealing and load distributing qualities are produced through elasticity of the circumference of the device. In a preferred embodiment, this elasticity exists within a range with an upper and lower limit.

At the upper elastic limit elastic aspect is stretched tight and the inside diameter cannot exceed a desired maximum through normal, anatomically generated outward pressure. This upper limit diameter is sized such that normal anatomical forces exerted upon the GBP cannot force the proximal end flange of the GBP through the GBP fixation device circumference under any circumstances, thereby assuring the positional integrity of the proximal end of the GBP.

The lower limit is where the device maintains a fixed circular inside diameter in a relaxed state with no outward pressure. The value of the lower limit inside diameter dimension is such that the stomach is not occluded inside the GBP fixation device circumference in the absence of the GBP. In this way, the GBP may be removed or replaced, through the throat, without impeding the digestive function of the patient.

In another embodiment of the GBP fixation device, the inside diameter is fixed and non-elastic. The stoma created by this device is larger than the portion of the GBP which passes through its center, and therefore does not exert contact pressure of the stomach wall against the GBP. The fixed inside diameter of the GBP fixation device is smaller than the proximal flange of the GBP such that the flange cannot pass through the middle of the fixation device. In this embodiment the flange seals against the floor of the stomach pouch (the interior of the stomach wall above the superior surface of the fixation device) due to pressure exerted by the flange against the floor created by pressure within the gastric pouch and/or traction on the tail of the GBP generated by peristalsis.

In yet another embodiment, the elastic or fixed diameter GBP fixation device holds in place an embodiment of the GBP (disclosed in WO 03/094785) that does not include an elongated tube extending through the pylorus. In this particular embodiment the system comprising the GBP fixation device and the GBP without trans-pyloric tube results in a restrictive-only treatment comparable to conventional AGBs in function, but with the distinct improvement of having a perfectly sized stoma (integral to the GBP) that does not need adjustment.

In so providing a stable, leak-tight fixation for the proximal end of the GBP, all the physiological and therapeutic benefits of the GBP can be realized.

A distinct, corollary invention to the use of a conventional GBP in conjunction with the new GBP fixation device described above is the invention of an improved, modified GBP adapted for fixation using a conventional AGB. (Lap-Band™—Inamed, Inc., Swedish Band™—Johnson and Johnson Corp., etc.) In this invention, the proximal end of the GBP would be adapted to conform, through the stomach wall, with the inherent shape of the cross section of a specific AGB design. Leak-tight seal control is accomplished through control of saline pressure normally used to control the size of the stoma. (Normally, in the absence of a GBP, the stoma of an AGB patient is visualized endoscopically and saline is injected through a subcutaneous port to obtain the desired stoma size. This can be characterized as volumetric control with visual feedback.) When used for fixation of a GBP, the pressure exerted on the stomach wall between the AGB and the GBP is the primary concern for maintenance of the leak-tight seal and avoidance of contact pressure necrosis. Therefore, saline will be injected using a device (i.e. syringe) equipped with means (i.e. gauge) for monitoring pressure. The pressure value will be a function of the inflation characteristics of the specific AGB design and the area presented by the inflation ring. Once again, in so providing a stable, leak-tight fixation for the proximal end of the GBP, all the physiological and therapeutic benefits of the GBP can be realized.

A further distinct invention made possible by the prior invention is a new therapeutic methodology for the treatment of obesity that allows the caregiver to react to the individual response of the patient.

Due to many factors beyond the control of the caregiver, individual patient response to obesity treatment is subject to great variability.

As previously noted, more aggressive weight loss can be expected for RGB than from AGB treatment for obesity and, since GBP is designed to mimic the response of RGB, we can expect GBP to also be more aggressive than AGB.

With the combinational AGB/GBP invention described above, the care giver will have the ability to switch between more and less aggressive weight loss treatments by simply implanting and removing the GBP.

For example, an obese patient with a life threatening concomitant condition, such as type II diabetes may be implanted with the AGB/GBP combination to facilitate rapid, early weight loss. If however, this same patient achieves satisfactory weight loss, but subsequently suffers from nutritional deficiencies due to the malabsorptive aspect of the GBP, the caregiver may remove the GBP through a simple, peroral endoscopic procedure. This same patient may later experience undesirable weight gain and could again be fitted with a GBP through peroral endoscopy, and so on.

Other objects, features and advantages will be apparent from the following detailed description of the preferred embodiments thereof taken in conjunction with the accompanying drawings:

### BRIEF DESCRIPTION OF THE DRAWINGS

FIG. 1 is a cross section view of a stomach with a GBP affixed using a band-like GBP fixation device.

FIG. 2 is an isometric view of a band-like GBP fixation device prior to implantation.

FIG. 3 is a detail view of a cross section view of a stomach with a specially adapted GBP affixed using a commercially available AGB.

FIG. 4 is a detail of a cross section of a specially adapted GBP for fixation using an AGB.

FIGS. 4A and 4B show exemplary forms of reinforcement for the GBP of the invention.

FIG. 5 is an isometric section view of a GBP and GBP fixation device system where the GBP is a restrictive-only class of device.

### DETAILED DESCRIPTION OF THE PREFERRED EMBODIMENTS

The present invention provides an improved gastric bypass prosthetic (GBP) device and an improved GBP fixation device, as well as a GBP kit for the surgical treatment of obesity. In one form, a gastric bypass prosthetic (GBP) device of the invention includes a flexible tube extending along a central axis from a proximal end to a distal end, and having a tubular tissue fixation portion at or near the proximal end.

The tissue fixation portion extends along the central axis between a proximal end and a distal end of the tissue fixation portion, and has an annular outer surface between the proximal end and said distal end of the tissue fixation portion.

In another form of the invention, a gastric bypass prosthetic (GBP) device includes a flexible tissue fixation portion extending along a central axis between a proximal end and a distal end of the tissue fixation portion, without a flexible tube extending therefrom. The device has an annular outer surface between the proximal end and the distal end of the tissue fixation portion. Preferably, the annular outer surface is concave, and may be smooth, textured, or have raised features.

In one form, the tissue fixation portion has a stoma element extending from an inner surface thereof, and the inner surface is characterized by a radius which monotonically decreases from the proximal end to the stoma element of the tissue fixation portion. Preferably, the stoma element is removable/insertable in the GBP device.

In one form, the proximal end of the tissue fixation portion is toroidal, and includes a circumferentially extending reinforcement element embedded therein, for example, a biocompatible coiled metallic spring.

The proximal end of the toroidal tissue fixation portion may have a hollow central region, which may be selectively inflated in vivo with a fluid medium, such as saline.

In yet another form of the invention, a gastric bypass prosthetic (GBP) kit includes a gastric bypass prosthetic (GBP) device of any of the above described forms, and a gastric bypass prosthetic (GBP) fixation device. Preferably, the GBP fixation device is an adjustable gastric banding (AGB) device, and most preferably, is a device of the above-described forms.

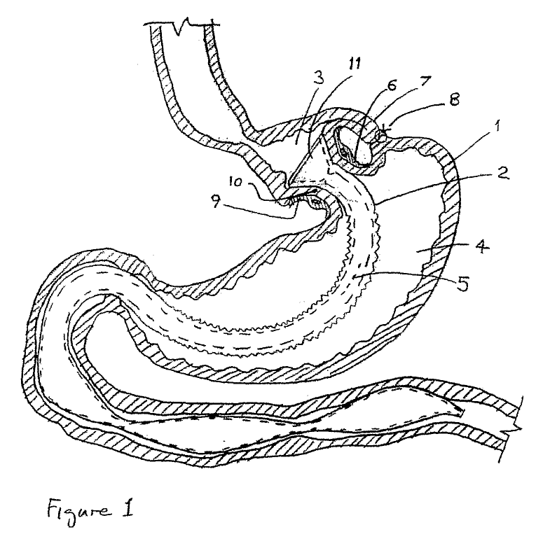

FIG. 1 shows a preferred embodiment of a band-like GBP fixation device used to affix a conventional (as disclosed) GBP in a stomach. A cross section of a stomach 1 with a GBP 2 implanted such that the stomach is divided into two chambers, a proximal chamber 3 and a distal chamber 4. The proximal chamber 3 is in fluid communication with the inner lumen of the GBP 5. The stomach 1 has been encircled by a band-like GBP fixation device 6. A portion of the stomach fundus 7 has been folded over the band-like GBP fixation device 6 and secured by one or more stitches 8 to prevent the band-like GBP fixation device 6 from slipping. The band-like GBP fixation device 6 exerts sufficient pressure to create a leak-tight seal between the inner lumen of the stomach and the GBP, yet insufficient pressure to cause formation of contact pressure necrosis or ulcer. The band-like GBP fixation device 6 includes an atramatic surface 9 that conforms to the shape of the stomach wall covering the GBP 10 and distributes tissue-contacting force over a relatively large area to minimize contact force per unit area (pressure). In a preferred embodiment the a band-like GBP fixation device is elastic such that its inside diameter stretches to accommodate variations in stomach wall thickness while still maintaining a leak-tight seal and without tissue injury. With the GBP removed (not shown) the elasticity of the band shrinks to a minimum (relaxed) diameter that is still sufficiently large to permit passage of food swallowed by the patient. The band-like GBP fixation device has sufficient hoop strength to resist passage of the annular element (flange) 11 of the proximal end of the GBP through the inside diameter, even under maximum anatomical loads.

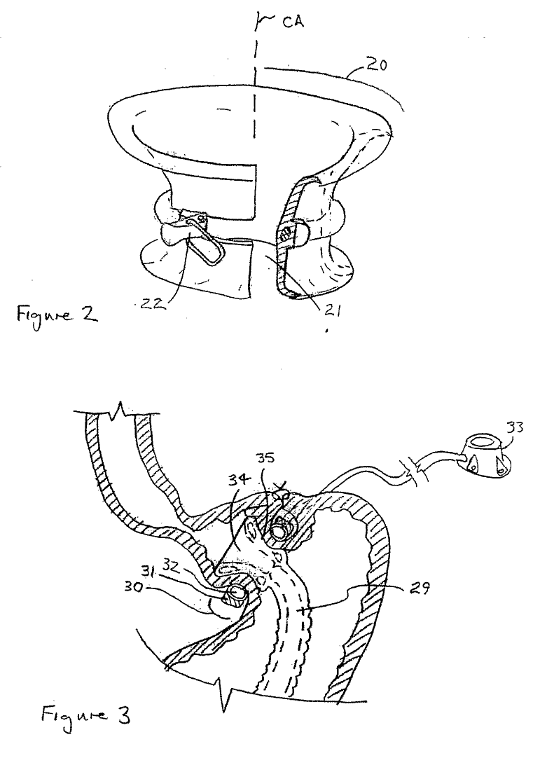

FIG. 2 shows a representative preferred embodiment of a band-like GBP fixation device. The body 20 is made from a biocompatible, atraumatic, non-irritating material (e.g. Dow Corning Corp. Silastic™ silicone rubber). In one preferred embodiment the material is reinforced with fabric for tear resistance. Still other embodiments are reinforced with a spun material such as Lycra™ to control elastic properties. The device is split 21 to allow it to be wrapped around the stomach and includes a fastening means 22 to allow it to be joined into a continuous ring within the body. In one preferred embodiment (shown) the fastening means 22 comprises a latching device. Other embodiments (not shown) employ hook and loop fasteners (e.g. Velcro™) snaps, cable ties or any other appropriate fastening means known to the art. Other embodiments (not shown) employ an inflatable bladder in place of, or in combination with elasticity to control contact pressure against the GBP.

FIG. 3 shows a commercially available AGB used to affix a specially adapted GBP 29 into the proximal end of a stomach. The AGB 30 includes a rigid band 31 fitted with an inflation membrane 32 on its inner surface. Inflation of the membrane 32 is controlled by injecting saline into a subcutaneous port 33. The proximal end 34 of the specially adapted GBP 29 has a contoured outer surface 35 to conform to the shape of the inflation membrane 32 to distribute contact pressure to prevent tissue injury and to maintain a leak-tight seal.

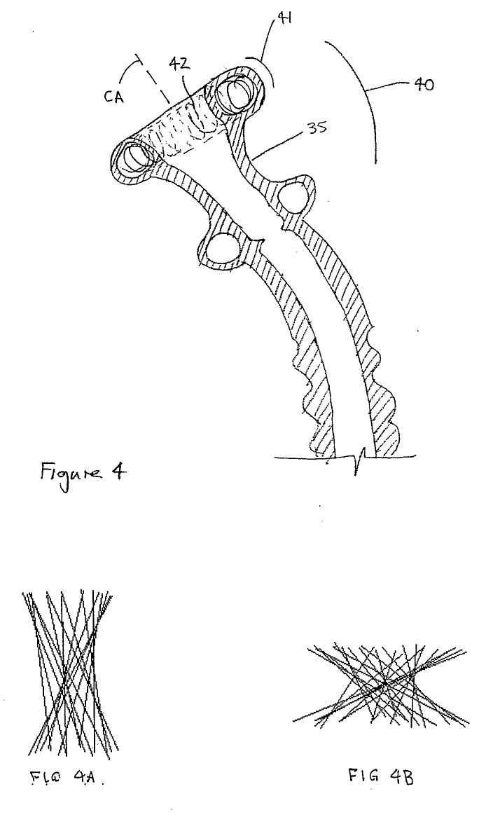

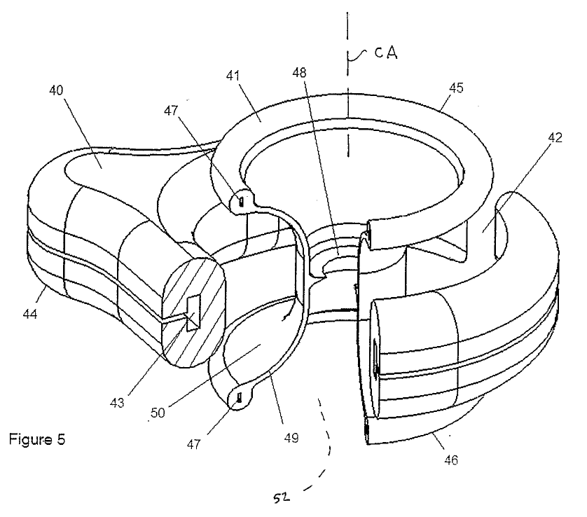

FIG. 4 shows a cross section detail of the proximal end of a GBP specially adapted for fixation using a commercially available AGB. The proximal end of the GBP 40 includes a concave, contoured surface 35. An annular element 41 is deployed at the proximal end of the GBP. The annular element 41 is constructed such that it prevents passage through the AGB under normal anatomical forces. It is, however, sufficiently flexible to be compacted into a trans-esophageal introducer tube for implantation and explanation through the throat. In one preferred embodiment these characteristics are achieved through reinforcement of the annular element with a coiled biocompatible metallic spring 42. Other embodiments include a funnel or hourglass shaped stent-like reinforcement cage made of a material such as superelastic Nitinol, for example, as shown in FIG. 4A. FIG. 4A shows the cage in a distorted state (small diameter) for implantation and FIG. 4B shows the cage in a “natural”/relaxed (large diameter) state as deployed. Other embodiments (not shown) inflate the hollow annular element in situ with saline or other fluids using endoscopic access upon implantation and deflate the annular element for ease of removal at explanation. Still other embodiments construct the annular element from elastomeric materials of the appropriate durometer to produce the desired stiffness and flexural characteristics. In all these embodiments the annular element is collapsed for introduction through the esophagus and expanded once in the proximal stomach or pouch. In some embodiments the annular element is designed to be re-collapsed and pulled into a tube for explanation. In other embodiments the annular element is designed to be cut into smaller pieces in situ for extraction through the esophagus. In some preferred embodiments the concave, contoured surface 35 is smooth. In other embodiments (not shown) the concave, contoured surface 35 includes a texture or raised features such as ridges or bumps to prevent loss of blood flow in the stomach wall or allow nutrient seepage into otherwise covered tissue surfaces.

FIG. 5 shows a system comprising a GBP fixation device 40 and a special class of GBP 41 that is restrictive-only, that is without a tube so that the principle effect is to restrict passage of food. The GBP fixation device 40 is split 42 to allow in situ encirclement of the stomach. GBP fixation device 40 is illustrated with an intermediate portion 52 cut away in order to allow a clear view of GBP device 41. This embodiment includes a channel 43 for a fastener band (e.g. Nylon cable tie, not shown) and a hollow bulge 44 designed to enclose the hard edges of the fastener band. The special restrictive-only GBP 41 includes both proximal 45 and distal 46 annular elements of sufficient size and rigidity to resist dislodgement by gastrointestinal muscular forces. In this embodiment, rigidity is enhanced and controlled by proximal and distal rings of reinforcing material 47. In a preferred embodiment these rings are welded strips of superelastic nitinol. This arrangement allows the annular elements to be twisted, compacted, folded or otherwise distorted to fit into an introducer tube for implantation through the esophagus. The GBP 41 also includes a molded stoma element 48 with optimum size and flex characteristics, therefore eliminating the need for stoma adjustment necessary with adjustable gastric bands without an artificial stoma. The relatively thin wall section 49 and smooth contour of the tissue contacting surfaces 50 of the molded elastomer GBP prevent gastric tissue damage such as contact pressure necrosis.

In a preferred form of the invention, the stoma element 48 of the GBP device 41 is removable/insertable. With that configuration, the device 41 can be positioned as desired in the stomach, and a GBP fixation device (such as device 41, or a band or other form of fixation device) can be placed exterior to the stomach, overlying the GBP device 41. Then, a toroidal stoma element 48 can be inserted, and preferably snap-fit, to the interior of the GBP device 41, for example as shown in FIG. 5. The removable/insertable stoma element 48 can be selected to have a specific geometry, e.g. defining a desired central opening (to effect a desired restriction in flow-through of food) and a desired outer diameter (to effect a desired compressive force between the stoma circumferential edge and the inner portion of the fixation device). The thin wall section 49 and resilient nature of the GBP device 41 permits use of a relatively broad range of diameters for the stoma element 48.

A similar type of removable/insertable stoma element is used in other forms of the invention, including but not limited to the forms illustrated in FIGS. 1, 3 and 4.

Those of skill in the art will recognize that the invention may be embodied in other specific forms without departing from the spirit or essential characteristics thereof. The presently described embodiments are therefore to be considered in all respects as illustrative and not restrictive, the scope of the invention being indicated by the appended claims rather than by the foregoing description, and all variations of the invention which are encompassed within the meaning and range of equivalency of the claims are therefor intended to be embraced therein.

1. A gastric bypass prosthetic (GBP) device comprising:
a flexible tube extending along a central axis from a proximal end to a distal end, and having a tubular tissue fixation portion at or near said proximal end,
wherein said tissue fixation portion extends along said central axis between a proximal end and a distal end of said tissue fixation portion, and has an annular outer surface between said proximal end and said distal end of said tissue fixation portion.

2. A GBP device according to claim 1, wherein said annular outer surface is concave.

3. A GBP device according to claim 2, wherein said concave outer surface is smooth.

4. A GBP device according to claim 2 wherein said concave outer surface is textured.

5. A GBP device according to claim 2 wherein said concave outer surface bears raised features.

6. A GBP device according to claim 1 wherein said tissue fixation portion has a stoma element extending from an inner surface thereof, and wherein said inner surface is characterized by a radius which monotonically decreases from said proximal end to said stoma element of said tissue fixation portion.

7. A GBP device according to claim 6 wherein said stoma element is removable/insertable in said GBP device.

8. A GBP device according to claim 1 wherein said tissue fixation portion is resilient and is adapted to permit being radially compacted sufficiently to fit within a trans-esophageal intraducer tube.

9. A GBP device according to claim 1 wherein said proximal end of said tissue fixation portion is toroidal.

10. A GBP device according to claim 9 wherein said toroidal tissue fixation portion includes a circumferentially extending reinforcement element embedded therein.

11. A GBP according to claim 10 wherein said reinforcement element is a stent-like cage.

12. A GBP according to claim 11 wherein said reinforcement element is made of Nitinol.

13. A GBP according to claim 10 wherein said reinforcement element is a ring.

14. A GBP according to claim 13 wherein said reinforcement element is made of Nitinol.

15. A GBP device according to claim 10 wherein said reinforcement element is a coiled metallic spring.

16. A GBP device according to claim 15 wherein said coiled metallic spring is biocompatible.

17. A GBP device according to claim 9 wherein said proximal end of said toroidal tissue fixation portion has a hollow central region.

18. A GBP device according to claim 17 wherein said toroidal fixation portion is adapted whereby said hollow central region may be selectively inflated in vivo with a medium.

19. A GBP device according to claim 18 wherein said medium is a fluid.

20. A GBP device according to claim 19 wherein said fluid is saline.

21. A gastric bypass prosthetic (GBP) device comprising:
a flexible tissue fixation portion extending along a central axis between a proximal end and a distal end of said tissue fixation portion, and having an annular concave outer surface between said proximal end and said distal end of said tissue fixation portion.

22. A GBP device according to claim 21, wherein said concave outer surface is smooth.

23. A GBP device according to claim 21 wherein said concave outer surface is textured.

24. A GBP device according to claim 21 wherein said concave outer surface bears raised features.

25. A GBP device according to claim 21 wherein said tissue fixation portion has a stoma element extending from an inner surface thereof, and wherein said inner surface is characterized by a radius which monotonically decreases from said proximal end to said stoma element of said tissue fixation portion.

26. A GBP device according to claim 25 wherein said stoma element is removable insertable in said GBP device.

27. A GBP device according to claim 21 wherein said tissue fixation portion is resilient and is adapted to permit being radially compacted sufficiently to fit within a trans-esophageal intraducer tube.

28. A GBP device according to claim 21 wherein said proximal end of said tissue fixation portion is toroidal.

29. A GBP device according to claim 28 wherein said toroidal tissue fixation portion includes a circumferentially extending reinforcement element embedded therein.

30. A GBP according to claim 29 wherein said reinforcement element is a stent-like cage.

31. A GBP according to claim 30 wherein said reinforcement element is made of Nitinol.

32. A GBP according to claim 29 wherein said reinforcement element is a ring.

33. A GBP according to claim 32 wherein said reinforcement element is made of Nitinol.

34. A GBP device according to claim 29 wherein said reinforcement element is a coiled metallic spring.

35. A GBP device according to claim 34 wherein said coiled metallic spring is biocompatible.

36. A GBP device according to claim 28 wherein said proximal end of said toroidal tissue fixation portion has a hollow central region.

37. A GBP device according to claim 36 wherein said toroidal fixation portion is adapted whereby said hollow central region may be selectively inflated in vivo with a medium.

38. A GBP device according to claim 37 wherein said medium is a fluid.

39. A GBP device according to claim 24 wherein said fluid is saline.

40. A gastric bypass prosthetic (GBP) fixation device comprising:
a flexible, resilient, split ring element disposed along a central axis having an axially extending inner surface and an axially extending outer surface, and extending circumferentially between opposed ends, said opposed ends forming a split.

41. A GBP fixation device according to claim 40, further comprising a selectively operable coupler assembly for coupling said opposed ends and biasing said opposed ends toward each other.

42. A GBP fixation device according to claim 41, wherein said coupler assembly includes a first portion affixed to said ring element at or near a first of opposed ends and a second portion affixed to said ring element at or near a second of said opposed ends, said first and second portions being adapted for selective engagement and said coupling and biasing.

43. A GBP fixation device according to claim 41 wherein coupler assembly includes an elongated tie element and a circuimferential groove for receiving said tie.

44. A GBP fixation device according to claim 41 wherein said coupler assembly is a hook and loop fastener.

45. A GBP fixation device according to claim 41 wherein said coupler assembly is a snap fastener.

46. A GBP fixation device according to claim 41 wherein said coupler assembly includes an eyelet on each of said opposed ends, said eyelets being adapted to receive a suture loop.

47. A GBP fixation device according to claim 40 wherein said ring element is biocompatible.

48. A GBP fixation device according to claim 47 wherein said ring element is composed of silicone rubber.

49. A GBP fixation device according to claim 31 wherein said ring element includes a reinforcement element embedded therein.

50. A GBP according to claim 49 wherein said reinforcement element is a stent-like cage.

51. A GBP according to claim 50 wherein said reinforcement element is made of Nitinol.

52. A GBP according to claim 49 wherein said reinforcement element is a ring.

53. A GBP according to claim 52 wherein said reinforcement element is made of Nitinol.

54. A GBP fixation device according to claim 49 wherein said reinforcement element is a fabric.

55. A GBP fixation device according to claim 54 wherein said reinforcement element is a spun material.

56. A GBP fixation device according to claim 40 wherein said ring element includes a circumferentially extending hollow region.

57. A GBP fixation device according to claim 56 wherein said hollow region may be selectively inflated in vivo with a medium.

58. A GBP fixation device according to claim 57 wherein said medium is a fluid.

59. A GBP fixation device according to claim 58 wherein said fluid is saline.

60-62. (canceled)
